(19) United States
(12) Patent Application Publication
(10) Pub. No.: US 2018/0361019 A1
(43) Pub. Date: Dec. 20, 2018

(54) HYALURONIC ACID COMPOSITION FOR PENILE INJECTIONS

(71) Applicant: VIVACY INTERNATIONAL SA, GENEVE (CH)

(72) Inventor: Richard DIACAKIS, Paris (FR)

(73) Assignee: VIVACY INTERNATIONAL SA, GENEVE (CH)

(21) Appl. No.: 16/063,360

(22) PCT Filed: Dec. 16, 2016
(86) PCT No.: PCT/EP2016/081624

§ 371 Date: Jul. 18, 2018

(30) Foreign Application Priority Data
Dec. 16, 2015 (EP) PCT/EP2015/080110

(51) Int. Cl. A61L 27/20

(52) U.S. Cl. CPC A61L 27/20; A61L 2400/06; A61L 2430/34

(57) ABSTRACT

A penis size enlargement and to hyaluronic acid compositions for use in penis injections. It also relates to penoplasty by injection of hyaluronic acid. It relates more particularly to a composition including at least one crosslinked hyaluronic acid, used in the treatment of locker room syndrome, characterized in that it is administered at a dose of at least 0.15 mL/cm2, and in that said composition is administered repeatedly, and a first administration is followed by n subsequent administration(s) spaced by a time interval from 6 and 20 months, where n≥1.

The present invention pertains to the field of penis size enlargement and to hyaluronic acid compositions for use in penis injections.

It also relates to penoplasty by injection of hyaluronic acid.

Since time immemorial, penis size and scrotum size have been a source of preoccupation for males.

In fact, representations of penises of disproportionate size have been associated in the past in particular with prominent persons: gods, statesmen, soldiers, explorers, heroes, etc.

The phallic symbolism, which has been present in cultures since Antiquity, relates to virility and to fecundity. In psychoanalysis, it is a fundamental symbolic element of construction of the subject.

Numerous attempts have been made with the aim of enlarging the size of the erect or non-erect penis.

For example, in primitive tribes, some men attached increasingly heavy weights to the penis. Although large sizes were obtained (more than 40 cm), these weights damaged the structure of the penis, making it less sensitive and incapable of having an erection. This empirical approach was therefore gradually abandoned.

The other approach that was developed was the introduction of material inside the penis to enlarge its volume.

One of the first techniques to be used was the implantation of adipose tissue in the penis, in particular the injection of fat from liposuction, from the abdominal wall or from the thigh, in the Dartos fascia under the penile skin. In a certain era, this was even the most commonly used technique.

A variant consisted in grafting dermis and fat inside the penis.

These techniques have a certain number of disadvantages (resorption of the fatty implant, irregularities, risk of infection, serious intervention, further surgery often necessary, necessity of taking the adipose tissue from an individual in a healthy zone and thus of making incisions, etc.).

In addition, permanent implants were developed, implants which required serious surgical interventions.

More recently, resorbable or permanent filler products began to be used, among which one can distinguish:
rapidly resorbable products having an effect that can last from several weeks to several months (for example, compositions based on hyaluronic acid or on collagen);
semi-permanent products having, for example, an action of 6 months to 1 year (for example, compositions based on modified hyaluronic acid and on polyvinyl alcohol);
permanent products or long-term filler products (for example, silicone-based implants).

These different techniques are extremely important in particular in that they make it possible to treat pathologies such as penis body dysmorphism disorder and in particular the locker room syndrome formerly called genital dysmorphophobia and currently called BDD (Penis Body Dysmorphic Disorder).

The permanent silicone-based products have well-described disadvantages.

We will mention here the inflammatory reactions which can cause the formation of granulomas, clusters of inflammatory cells, several months after the injection of silicone. Another commonly mentioned risk is that of migration of the implant due to non-assimilation by the tissues.

These side effects and the absence of a surgical procedure are arguments in favor of resorbable products such as hyaluronic acid, for example.

The penis has two main regions: the glans penis and the penis body. These zones are very different morphologically, to such an extent that a composition suitable for one of these two regions is only very rarely suitable for the other of these two regions.

The term “penis body” is used to denote the region of the penis over the length of which the erectile tissues 10 of the corpus cavernosum penis and the fasciae extend. The penis body has a very particular structure which is illustrated in FIG. 1.

The term “glans penis” is used to denote the region of the penis corresponding to the end of the penis, characterized in particular by the absence of the erectile tissues 10 of the corpora cavernosa penis and of the fasciae.

Hyaluronic acid has been used for more than fifteen years in the field of aesthetic medicine, where its safety and efficacy have been proven. To date, in the market of filler gels for aesthetic purpose or “fillers”, gels based on crosslinked hyaluronic acid produced by biofermentation are the most commonly used products.

Among the medical applications, we will mention, for example, injections for replacing deficient biological fluids, for example, in the joints in order to replace the synovial fluid, post-surgical injection in order to prevent peritoneal adhesions, periurethral injections for treating incontinence, and injections after presbyopia surgery.

Among the aesthetic applications, mention will be made, for example, of injections for filling wrinkles, small wrinkles and cutaneous defects or for enlarging volumes, for example, of the lips, of the cheekbones, etc.

The use of hyaluronic acid produced by biofermentation in fields such as the filling of wrinkles, viscosupplementation, ophthalmic treatment or the treatment of urinary incontinence has been all the more recognized and appreciated given its natural presence in the human body and more particularly in the dermis, the synovial fluid and the vitreous fluid; the risks due to the side effects are minimized.

Numerous patent applications or publications have been filed or published on compositions based on hyaluronic acid including, in addition to hyaluronic acid, active substances or excipients in order to modify or improve the properties of the composition as a function of the particular applications.

For example, the application WO 2013/186493 discloses compositions of hyaluronic acid including a sucrose octasulfate, and the application WO 2014/032804 discloses hyaluronic acid compositions including a vitamin C derivative.

Compositions based on hyaluronic acid and including a polyol have been also described in the prior art.

For example, in the application WO 2007/077399 in the name of ANTEIS, compositions for dermatological use based on hyaluronic acid or one of the salts thereof and a polyol are presented.

Certain patent applications and publications thus relate to compositions based on hyaluronic acid and including a local anesthetic.

The application WO 93/12801 in the name of REINMULLER describes gels for treating wounds and keloid scars by subcutaneous injection. Example 1 of this application relates to a composition based on hyaluronic acid of the HYLAGEL® (company BIOMATRIX) type and containing lidocaine.

The article by WAHL, G. in Journal of Cosmetics Dermatology relates to the incorporation of lidocaine in filler compositions based on hyaluronic acid. The results presented pertain to tests carried out using JUVEDERM ULTRA® which is a filler product based on crosslinked hyaluronic acid. According to this article, more than 87% of the patients reported having less pain during the injection of compositions incorporating lidocaine.

Compositions based on hyaluronic acid including at the same time mannitol and lidocaine are marketed, this is the case, for example, of the STYLAGE® product line marketed by VIVACY.

In the application WO 2014/123408 in the name of KIRCH UROLOGY, a permanent penile implant is presented. It is intended for filling an internal portion of the pubis after cutting the suspensory ligaments. It is thus intended for implantation consecutively to or concomitantly with a surgical intervention.

In the application FR 2 951 368 in the name of Jacques DERHY, other permanent implants are presented. They are in the form of strips which, when implanted, enlarge in particular the penile girth. Here again, the necessity of a surgical intervention is obvious.

A comparative study between filling with fat and filling with hyaluronic acid (Macrolane® from Q-MED) is presented in the reference “Use of Macrolane® VRR30 in Hemicirconferential Penis Enlargement” (Aesthetics Surgery Journal 2013; 33: 258). In this study, the filler products are injected hemicircumferentially in the dorsal portion of the penis. The conclusion drawn is that the composition based on hyaluronic acid is superior in terms of duration of operation, patient satisfaction, size enlargement, complications as well as durability.

In the study described in the publication “Complications of Penis or Scrotum Enlargement due to Injections with Permanents Filing Substances” (Dermotol Surg 2012; 38: 1244-1250), certain complications connected with the use of permanent implants based on silicone oil and on polyalkylimide are described. The article concludes that it is not recommended to use such permanent products.

However, the use of resorbable products presents a disadvantage in that it could lead to difficult situations between two injections, when the resorption is highest.

The publication KWAK T I ET AL: “The Effects of Penile Girth Enhancement using Injectable Hyaluronic Acid Gel, a Filler,” JOURNAL OF SEXUAL MEDICINE, Vol. 8, No. 12, 2011, pages 3407-3413, XP009190934, relates to a study on the enlargement of the circumference of the penis by injection of hyaluronic acid; in it, it is concluded that the method using hyaluronic acid is minimally invasive and has long-term efficacy (18 months after the single injection, which can be corrected shortly after if this single injection is not adequate).

The publication J KIM ET AL: “Human glans penis augmentation using injectable hyaluronic acid gel,” INTERNATIONAL JOURNAL OF IMPOTENCE RESEARCH., Vol. 15, No. 6, 2003, pages 439-443, XP055288691, relates to a method of glans size enlargement by a single injection of hyaluronic acid; the injection is thus administered into the glans penis.

### DESCRIPTION OF THE FIGURES

FIG. 1: Simplified Perspective View of the Structure of the Penis Body.

FIG. 1 is a perspective and cross-sectional view of a penis body 1, in which it can be seen that its structure consists, from outside to inside, of the skin 2, the Dartos fascia 6, the Buck's fascia 7, the external wall of the tunica albuginea 11, and the erectile tissues 10 of the corpora cavernosa penis.

The body of the penis 1 is supplied by the dorsal artery 5, and the dorsal veins 3 and 4 as well as the spongy body 8 are also represented,

In this figure, the particularly preferred zones of injection are made visible, namely in the penis body, either between the Dartos fascia 6 and the Bucks fascia 7, or between the Bucks fascia 7 and the external wall of the tunica albuginea 11.

FIG. 2a: Diagrammatic View of the Penis with Representations of the Zones of the Penis Body 1.

FIG. 2a is a diagrammatic view of a penis 100 seen from the top, in which the penis body 1 is differentiated from the glans penis 4; also represented is the pubic base of the penis 2.

In the portion pertaining to the penis body 1, eight zones A, B, C, D, E, F, G, and H of administration are represented, all located on the dorsal side 200 of the penis.

FIG. 2b: Diagrammatic Front View of the Penis with Representation of the Extent of the Zones of Administration.

FIG. 2b is a diagrammatic cross-sectional view of the penis body 1 of a penis 100, in which the dorsal side 200 and the ventral side 300 of the penis 100 are represented. The cross-hatched zone corresponds to the transverse extent of the zones in which the administration can take place, from approximately 4 hours to approximately 8 hours.

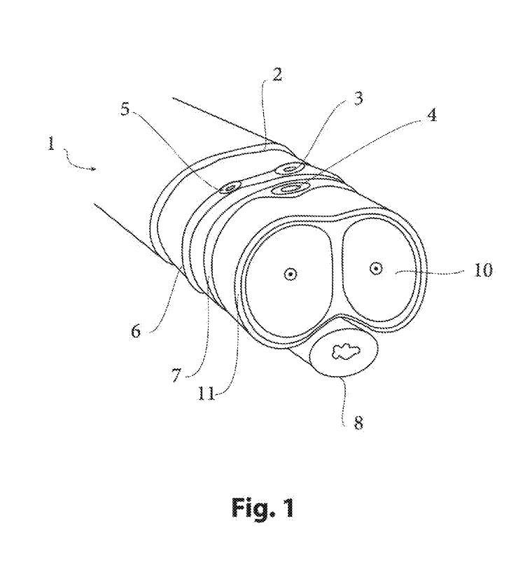

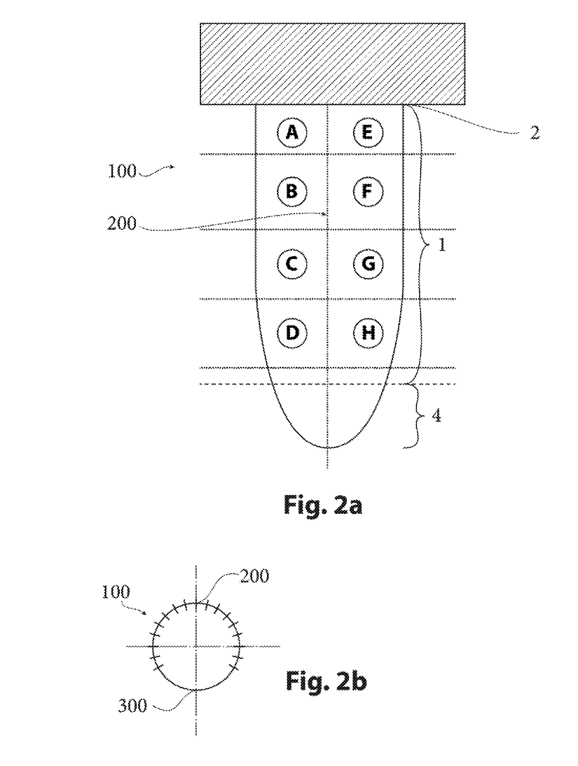

The invention relates to a composition comprising at least one crosslinked acid, used in the treatment of locker room syndrome and having certain features described below.

Surprisingly, it was shown by the applicant that the repetition of the injections had the effect of creating a kind of implant that persists over time, making it possible to inject less and less hyaluronic acid and/or inject at longer intervals, while keeping the same volume enlarging effect. Thus, although a possible 20 to 30% decrease of the result after 2 years has been observed with the compositions according to the invention, this decrease in the volume enlarging effect is clearly less than what is normally observed, resulting in the long run in constancy of the effect. In addition, an increasing duration of the effect has been observed in cases of repeated treatments.

Surprisingly, it was shown that the administration by injection of a particular dose of at least one hyaluronic acid made possible a penis size enlargement and an improved long-term effect without complete resorption by means of repeated administrations.

In particular, it was observed surprisingly that, when the composition according to the invention was administered at a dose of approximately 0.15 mL/cm², the volume injected gave rise to a reaction of the organism, having the effect of producing an individual palpable mass, delimited, as if encapsulated, thus protecting the hyaluronic acid from excessively quick degradation. The persistence of the hyaluronic acid under these conditions is thus longer.

In addition, it has been noted that these effects are observed even more particularly when the injections occur in contact with the fasciae, that is to say either between the Dartos fascia 6 and the Buck's fascia 7, or between the Buck's fascia 7 and the external wall of the tunica albuginea 11.

Without wishing to be bound to any one explanation, it seems that this is due to a colonization of fibers originating from fascia(e) around the gel.

Finally, it has been observed even more surprisingly that, when the two above-mentioned aspects are combined, that is to say a particular dose administered and repeated injections, then the effects are potentiated, that is to say an enlargement of the penis size is achieved with a long-term effect, throughout the entire time of use.

Thus, in terms of durability and safety, the compositions according to the invention are capable of having a volume enlarging effect for several years, while avoiding the complications frequently associated with the use of permanent implants.

In addition, these compositions moreover have a certain number of other advantages.

With regard to the implantation, the compositions according to the invention are particularly easy to inject using syringes and needles conventionally used in the filling field, thus avoiding any serious operation and consequently any complication, any risk of infection, etc.

With regard to the immediateness of the effect, the compositions according to the invention provide a nearly instant size enlargement as well as an appreciable effect of penis heaviness.

With regard to the improvement of the sex life, it is important that the compositions according to the invention do not interfere with the erection. In patients suffering both from both perceived insufficiency of size and erection disorders, it has been observed that the compositions according to the invention improve the duration, intensity and rapidity of onset of the erection. In addition, sexual relations can be resumed at the earliest 24 hours after the injection.

With regard to the reversibility, the hyaluronic acid can be removed by suctioning again if the patient so wishes.

The term “hyaluronic acid” is used to denote hyaluronic acid alone or in a mixture, hyaluronic acid which has been optionally chemically modified by substitution, alone or in a mixture, optionally in the form of one of the salts thereof, alone or in a mixture. In the context of the present invention, the composition includes at least one crosslinked hyaluronic acid.

The term “local anesthetic” is used to denote a local anesthetic or one of the salts thereof, alone or in a mixture.

In general, in the text of this application, the limits of a range of values are included in this range, in particular in the expression “from . . . to . . . .”

“Mw” or “molecular weight” or “average molecular weight” is used to denote the weight average molecular weight of the polymers, measured in daltons.

In the present invention, the crosslinking rate X is defined as being equal to the ratio:

$$X = \frac{( \begin{matrix}Number of moles of crosslinking agent introduced \\ into the reaction medium\end{matrix} )}{( \begin{matrix}Number of moles of disaccharide unit introduced \\ into the reaction medium\end{matrix} )}$$

The term “implantation” denotes an implantation performed during a session. Usually several implantations (which can be injections, for example, and more particularly subcutaneous injections) are carried out in a session. In the present invention, each implantation corresponds to a treated penis area of approximately 5 to 6 cm², and the injected volume is at least 1 mL, corresponding to an implanted volume of at least 1 mL/5 cm² or 1 mL/6 cm², or 0.15 to 0.2 mL/cm².

These sessions (each including one or more implantations) can be repeated.

When reference is made to the “total implanted/injected volume,” this refers to the total volume implanted/injected during a session, corresponding either to the single implantation/injection volume or to the sum of the volumes of the implantations/injections.

The invention relates to a composition including at least one crosslinked hyaluronic acid, which is used in the treatment of locker room syndrome, characterized:
in that it is administered at a dose of at least 0.15 mL/cm²;
   and/or
in that it is administered repeatedly, and a first administration is moreover followed by n subsequent administration(s) spaced by a time interval greater than or equal to 6 months, where n≥1.

The invention relates to a composition including at least one crosslinked hyaluronic acid, which is used in the treatment of locker room syndrome, characterized in that it is administered at a dose of at least 0.15 mL/cm².

The invention relates to a composition including at least one crosslinked hyaluronic acid, which is used in the treatment of locker room syndrome, characterized in that it is administered in a number of zones of the penis body 1 from 1 to 10.

The invention relates to a composition including at least one crosslinked hyaluronic acid, which is used in a method of subcutaneous penile implantation in order to increase the penile volume, characterized in that the implantation is carried out at a dose of at least 0.15 mL/cm².

The invention relates to a composition including at least one crosslinked hyaluronic acid, which is intended to be implanted in the penis of a patient, characterized in that the implantation is carried out at a dose of at least 0.15 mL/cm².

In an embodiment, the invention relates to a method of implantation of at least one composition including at least one crosslinked hyaluronic acid in the penis of a patient, characterized in that the implantation is carried out at a dose of at least 0.15 mL/cm².

In an embodiment, the dose is at least 0.2 mL/cm².

In an embodiment, the dose is at least 0.4 mL/cm².

In an embodiment, the dose is at least 0.8 mL/cm².

The invention also relates to a composition according to the invention, characterized in that said composition is administered repeatedly, and a first administration is moreover followed by n subsequent administration(s) spaced by a time interval greater than or equal to 6 months, where n≥1.

The invention also relates to a composition according to the invention, characterized in that said composition is administered repeatedly, and a first administration is moreover followed by n subsequent administration(s) spaced by a time interval greater than or equal to 6 months, where n≥1.

The invention also relates to a composition according to the invention, characterized in that said composition is administered repeatedly, and a first administration is moreover followed by n subsequent administration(s) spaced by a time interval from 6 to 30 months, where n≥1.

The invention also relates to a composition according to the invention, characterized in that said composition is administered repeatedly, and a first administration is moreover followed by n subsequent administration(s) spaced by a time interval from 6 to 25 months, where n≥1.

The invention also relates to a composition according to the invention, characterized in that said composition is administered repeatedly, and a first administration is moreover followed by n subsequent administration(s) spaced by a time interval from 6 to 20 months, where n≥1.

For example, if n=3 and the time interval between the administrations is 10 months, the patient receives a total of 4 administrations (n subsequent administrations+1 for the initial administration), the total duration between the day of the first administration and the day of the fourth and last administration is 30 months.

In an embodiment, the dose administered is administered in at least one region selected from the group of the penis body 1 and the glans penis 4.

In an embodiment, the dose administered is administered in the penis body 1.

In an embodiment, the dose administered is administered in the glans penis 4.

In an embodiment, the dose administered is administered in at least one zone of the penis body 1 having an area from 2 to 10 cm².

In an embodiment, the dose administered is administered in at least one zone of the penis body 1 having an area from 2 to 8 cm².

In an embodiment, the dose administered is administered in at least one zone of the penis body 1 having an area from 2 to 6 cm².

In an embodiment, the dose administered is administered in at least one zone of the penis body 1 having an area from 2 to 5 cm².

In an embodiment, the dose administered is administered in several zones of the penis body.

In an embodiment, the number of zones is from 1 to 10.

In an embodiment, the number of zones of the penis body is from 1 to 8.

In an embodiment, the implantation is carried out in at least one zone of the penis body 1 having an area from 2 to 10 cm².

In an embodiment, the implantation is carried out in at least one zone of the penis body 1 having an area from 2 to 8 cm².

In an embodiment, the implantation is carried out in at least one zone of the penis body 1 having an area from 2 to 6 cm².

In an embodiment, the implantation is carried out in at least one zone of the penis body 1 having an area from 2 to 5 cm².

In an embodiment, the implantation is carried out in a number of zones of the penis body 1 from 1 to 10.

In an embodiment, the implantation is carried out in a number of zones of the penis body 1 from 1 to 8.

In an embodiment, the implantation is carried out in 8 zones of the penis body 1.

In an embodiment, a first administration is moreover followed by n administration(s) spaced by a time interval from 6 to 15 months, where n≥1.

In an embodiment, a first administration is moreover followed by n subsequent administration(s) spaced by a time interval from 6 to 15 months, where n≥3.

In an embodiment, a first administration is moreover followed by n subsequent administration(s) spaced by a time interval from 6 to 15 months, where n≥4.

In an embodiment, the implantation is carried out by injection.

In an embodiment, the dose is administered by injection

In an embodiment, the injection is administered by means of an injection device selected from the group consisting of a needle and a cannula.

In an embodiment, the injection is administered by means of a needle.

In an embodiment, the injection is administered by means of a cannula.

In an embodiment, the injection is administered subcutaneously, between the corpora cavernosa and the skin. In an embodiment, the dose administered is administered at a depth selected from the group consisting of between the Dartos fascia 6 and the Buck's fascia 7, between the Buck's fascia 7 and the external wall of the tunica albuginea 11, or both.

In an embodiment, the dose administered is administered between the Dartos fascia 6 and the Buck's fascia 7.

In an embodiment, the dose administered is administered between the Buck's fascia 7 and the external wall of the tunica albuginea 11.

In an embodiment, the composition is characterized in that the at least one crosslinked hyaluronic acid has a crosslinking rate X from 0.001 to 0.5.

In an embodiment, the composition is characterized in that the at least one crosslinked hyaluronic acid has a crosslinking rate X from 0.01 to 0.4.

In an embodiment, the composition is characterized in that the at least one crosslinked hyaluronic acid has a crosslinking rate X from 0.1 to 0.3.

In an embodiment, the composition is characterized in that the at least one crosslinked hyaluronic acid has a crosslinking rate X of 0.06.

In an embodiment, the composition is characterized in that the at least one crosslinked hyaluronic acid has a crosslinking rate X of 0.07.

In an embodiment, the composition is characterized in that the at least one crosslinked hyaluronic acid has a crosslinking rate X of 0.12.

In an embodiment, the composition is characterized in that the molecular weight Mw of the at least one hyaluronic acid is in a range from 0.01 MDa to 5 MDa.

In an embodiment, the composition is characterized in that the molecular weight Mw of the at least one hyaluronic acid is in a range from 0.1 MDa to 3.5 MDa.

In an embodiment, the composition is characterized in that the molecular weight Mw of the at least one hyaluronic acid is in a range from 1 MDa to 3 MDa.

In an embodiment, the composition is characterized in that the molecular weight Mw of the at least one hyaluronic acid is in a range from 1 MDa to 2 MDa.

In an embodiment, the composition is characterized in that the molecular weight Mw of the at least one hyaluronic acid is 1 MDa.

In an embodiment, the composition is characterized in that the molecular weight Mw of the at least one hyaluronic acid is 2 MDa.

In an embodiment, the composition is characterized in that the molecular weight Mw of the at least one hyaluronic acid is 3 MDa.

In an embodiment, the composition is characterized in that the at least one crosslinked hyaluronic acid or one of the salts thereof, alone or in a mixture, is chemically modified by substitution.

In an embodiment, the composition is characterized in that the at least one hyaluronic acid is doubly crosslinked, as described in the patent application WO 2000/046253 in the name of Fermentech Medical Limited.

In an embodiment, the composition is characterized in that the at least one hyaluronic acid or one of the salts thereof, crosslinked, is a mixture of hyaluronic acids.

In an embodiment, the composition is characterized in that the at least one hyaluronic acid is a mixture of hyaluronic acids or one of the salts thereof, crosslinked.

In an embodiment, the composition is characterized in that the at least one hyaluronic acid is a mixture of hyaluronic acids or one of their salts, crosslinked, monophasic such as the one described in the patent application WO 2009/071697 in the name of the applicant.

In an embodiment, the mixture of hyaluronic acids or one of their salts, crosslinked, is a mixture obtained by mixing several hyaluronic acids or one of their salts, having different molecular weights before being crosslinked, as described in the patent application WO 2004/092222 in the name of Corneal Industrie.

In an embodiment, the composition is characterized in that the at least one hyaluronic acid is substituted with a group contributing lipophilic or hydrophilic properties, such as, for example, the substituted hyaluronic acids described in the patent application FR 2 983 483 in the name of the applicant.

In an embodiment, the composition is characterized in that at least one hyaluronic acid is in the form of a sodium or potassium salt.

In an embodiment, the composition is characterized in that at least one hyaluronic acid or one of the salts thereof is co-crosslinked.

In an embodiment, the composition is characterized in that it is selected from the group consisting of the formulations STYLAGE L®, STYLAGE XL®, STYLAGE XXL®, DESIRIAL®, DESIRIAL HOMME®, JUVEDERM 4®, SURGIDERM 30® and GLYTONE 4®.

In an embodiment, the composition is the formulation STYLAGE L®.

These commercial compositions are characterized by the fact that they are monophasic.

The formulation STYLAGE L® is a formulation including:
a mixture of monophasic crosslinked hyaluronic acids as described in the patent application WO 2009/071697, the total concentration of hyaluronic acid being 24 mg/mL, and the average degree of modification is 5%;
mannitol at a concentration of 30 mg/mL.

The formulation STYLAGE L® possesses the following rheological properties: module G′ (Pa, 1 Hz) from 210 to 270, module G″ from 34 to 40.

Particularly surprisingly, it was found that no migration of the implanted hyaluronic acid was observed when monophasic products were used, which are thus preferred.

In the context of the present application, “monophasic composition” is understood to mean a composition which includes no step of particle formulation in the production method thereof.

In an embodiment, the composition is characterized in that the at least one hyaluronic acid is a mixture of hyaluronic acids or one of the salts thereof, crosslinked monophasic.

In an embodiment, the composition is characterized in that the at least one crosslinked hyaluronic acid has a crosslinking rate X from 0.001 to 0.5, in that the molecular weight Mw of the at least one hyaluronic acid is in a range from 0.01 MDa to 5 MDa, and in that the concentration of the at least one hyaluronic acid [HA] is from 2 mg/g to 50 mg/g of total weight of said composition.

In an embodiment, the composition is characterized in that the at least one hyaluronic acid has an elastic component G′ (25° C., 1 Hz) from 220 to 260 Pa.

In an embodiment, the composition is characterized in that the at least one hyaluronic acid has an elastic component G′ (25° C., 1 Hz) of approximately 240 Pa.

In a preferred embodiment, the at least one hyaluronic acid includes:
at least one hyaluronic acid having a crosslinking rate X1 greater than 0.4;
at least one hyaluronic acid having a crosslinking rate X2 such that 0<X2<X1.

In an embodiment, said first and said second hyaluronic acid have an identical average molecular weight.

In an embodiment, the at least one first crosslinked hyaluronic acid has a crosslinking rate X1 greater than 0.45.

In an embodiment, the at least one first crosslinked hyaluronic acid has a crosslinking rate X1 from 0.4 to 0.8.

In an embodiment, the at least one first crosslinked hyaluronic acid has a crosslinking rate X1 from 0.4 to 0.5.

In an embodiment, the at least one second crosslinked hyaluronic acid has a crosslinking rate X2 from 0.01 to 0.2.

In an embodiment, the at least one second crosslinked hyaluronic acid has a crosslinking rate X2 from 0.05 to 0.12.

In an embodiment, the composition is characterized in that the concentration of the at least one hyaluronic acid [HA] is from 2 mg/g to 50 mg/g of total weight of said composition.

In an embodiment, the composition is characterized in that the concentration of the at least one hyaluronic acid [HA] is from 4 mg/g to 40 mg/g of total weight of said composition.

In an embodiment, the composition is characterized in that the concentration of the at least one hyaluronic acid [HA] is from 5 mg/g to 30 mg/g of total weight of said composition.

In an embodiment, the composition is characterized in that the concentration of the at least one hyaluronic acid [HA] is from 10 mg/g to 30 mg/g of total weight of said composition.

In an embodiment, the composition is characterized in that the concentration of the at least one hyaluronic acid [HA] is from 20 mg/g to 27 mg/g of total weight of said composition.

In an embodiment, the composition is characterized in that the concentration of the at least one hyaluronic acid [HA] is 20 mg/g of total weight of said composition.

In an embodiment, the composition is characterized in that the concentration of the at least one hyaluronic acid [HA] is 24 mg/g of total weight of said composition.

In an embodiment, the composition is characterized in that the concentration of the at least one hyaluronic acid is from 0.2 to 5% by weight with respect to the total weight of said composition.

In an embodiment, the composition is characterized in that the concentration of the at least one hyaluronic acid is greater than or equal to 1% by weight with respect to the total weight of said composition.

In an embodiment, the composition is characterized in that the concentration of the at least one hyaluronic acid [HA] is 20 mg/g of total weight of said composition.

In an embodiment, the composition moreover includes at least one non-crosslinked hyaluronic acid or one of the salts thereof, alone or in a mixture.

In an embodiment, the composition moreover includes at least one second crosslinked hyaluronic acid or one of the salts thereof, alone or in a mixture.

In an embodiment, the composition includes moreover at least one polyol.

In an embodiment, the composition is characterized in that the at least one polyol is selected from the group consisting of glycerol, sorbitol, propylene glycol, xylitol, mannitol, erythritol, maltitol and lactitol, alone or in a mixture.

In an embodiment, the composition is characterized in that the at least one polyol is selected from the group consisting of mannitol, sorbitol, maltitol and glycerol, alone or in a mixture.

In an embodiment, the composition is characterized in that the at least one polyol is selected from the group consisting of mannitol, sorbitol and maltitol, alone or in a mixture.

In an embodiment, the composition is characterized in that the at least one polyol is mannitol.

In an embodiment, the composition is characterized in that the at least one polyol is sorbitol.

In an embodiment, the composition is characterized in that the at least one polyol is maltitol.

In an embodiment, the composition is characterized in that the at least one polyol is glycerol.

In an embodiment, the composition is characterized in that said composition includes at least mannitol and sorbitol.

In an embodiment, the composition is characterized in that said composition includes at least mannitol and maltitol.

In an embodiment, the composition is characterized in that the concentration of the at least one polyol [Po] is from 0.01 mg/g to 50 mg/g.

In an embodiment, the composition is characterized in that the concentration of the at least one polyol [Po] is from 10 to 40 mg/g of total weight of said composition.

In an embodiment, the composition is characterized in that the concentration of the at least one polyol [Po] is from 15 to 30 mg/g of total weight of said composition.

In an embodiment, the composition is characterized in that the concentration of the at least one polyol [Po] is from 15 to 25 mg/g of total weight of said composition.

In an embodiment, the composition is characterized in that the concentration of the at least one polyol [Po] is from 20 to 40 mg/g of total weight of said composition.

In an embodiment, the composition is characterized in that the concentration of the at least one polyol [Po] is from 20 to 30 mg/g of total weight of said composition.

In an embodiment, the composition is characterized in that the concentration of the at least one polyol [Po] is from 25 to 35 mg/g of total weight of said composition.

In an embodiment, the composition is characterized in that the concentration of the at least one polyol [Po] is 35 mg/g of total weight of said composition.

In an embodiment, the composition is characterized in that the at least one polyol is mannitol and the concentration thereof is from 10 to 40 mg/g of total weight of said composition.

In an embodiment, the composition is characterized in that the at least one polyol is mannitol and the concentration thereof is from 15 to 30 mg/g of total weight of said composition.

In an embodiment, the composition is characterized in that the at least one polyol is mannitol and the concentration thereof is from 15 to 25 mg/g of total weight of said composition.

In an embodiment, the composition is characterized in that the at least one polyol is mannitol and the concentration thereof is from 20 to 40 mg/g of total weight of said composition.

In an embodiment, the composition is characterized in that the at least one polyol is mannitol and the concentration thereof is from 25 to 35 mg/g of total weight of said composition.

In an embodiment, the composition is characterized in that the at least one polyol is mannitol and the concentration thereof is 35 mg/g of total weight of said composition.

In an embodiment, the composition is characterized in that the at least one polyol is sorbitol and the concentration thereof is from 10 to 40 mg/g of total weight of said composition.

In an embodiment, the composition is characterized in that the at least one polyol is sorbitol and the concentration thereof is from 15 to 30 mg/g of total weight of said composition.

In an embodiment, the composition is characterized in that the at least one polyol is sorbitol and the concentration thereof is from 15 to 25 mg/g of total weight of said composition.

In an embodiment, the composition is characterized in that the at least one polyol is sorbitol and the concentration thereof is from 20 to 40 mg/g of total weight of said composition.

In an embodiment, the composition is characterized in that the at least one polyol is sorbitol and the concentration thereof is from 25 to 35 mg/g of total weight of said composition.

In an embodiment, the composition is characterized in that the at least one polyol is sorbitol and the concentration thereof is 35 mg/g of total weight of said composition.

In an embodiment, the composition is characterized in that the at least one polyol is maltitol and the concentration thereof is from 10 to 40 mg/g of total weight of said composition.

In an embodiment, the composition is characterized in that the at least one polyol is maltitol and the concentration thereof is from 15 to 30 mg/g of total weight of said composition.

In an embodiment, the composition is characterized in that the at least one polyol is maltitol and the concentration thereof is from 15 to 25 mg/g of total weight of said composition.

In an embodiment, the composition is characterized in that the at least one polyol is maltitol and the concentration thereof is from 20 to 40 mg/g of total weight of said composition.

In an embodiment, the composition is characterized in that the at least one polyol is maltitol and the concentration thereof is from 25 to 35 mg/g of total weight of said composition.

In an embodiment, the composition is characterized in that the at least one polyol is maltitol and the concentration thereof is 35 mg/g of total weight of said composition.

In an embodiment, the composition is characterized in that the at least one polyol is glycerol and the concentration thereof is from 10 to 40 mg/g of total weight of said composition.

In an embodiment, the composition is characterized in that the at least one polyol is glycerol and the concentration thereof is from 15 to 30 mg/g of total weight of said composition.

In an embodiment, the composition is characterized in that the at least one polyol is glycerol and the concentration thereof is from 15 to 25 mg/g of total weight of said composition.

In an embodiment, the composition is characterized in that the at least one polyol is glycerol and the concentration thereof is from 20 to 40 mg/g of total weight of said composition.

In an embodiment, the composition is characterized in that the at least one polyol is glycerol and the concentration thereof is from 25 to 35 mg/g of total weight of said composition.

In an embodiment, the composition is characterized in that the at least one polyol is glycerol and the concentration thereof is 35 mg/g of total weight of said composition.

In an embodiment, the composition is characterized in that the concentration of the at least one local anesthetic [LA] is from 0.01 mg/g to 50 mg/g of total weight of said composition.

In an embodiment, the composition is characterized in that the concentration of the at least one local anesthetic [LA] is from 0.05 mg/g to 45 mg/g of total weight of said composition.

In an embodiment, the composition is characterized in that the concentration of the at least one local anesthetic [LA] is from 0.1 mg/g to 40 mg/g of total weight of said composition.

In an embodiment, the composition is characterized in that the concentration of the at least one local anesthetic [LA] is from 0.2 mg/g to 30 mg/g of total weight of said composition.

In an embodiment, the composition is characterized in that the concentration of the at least one local anesthetic [LA] is from 0.5 mg/g to 20 mg/g of total weight of said composition.

In an embodiment, the composition is characterized in that the concentration of the at least one local anesthetic [LA] is from 1 mg/g to 15 mg/g of total weight of said composition.

In an embodiment, the composition is characterized in that the concentration of the at least one local anesthetic [LA] is from 1 mg/g to 10 mg/g of total weight of said composition.

In an embodiment, the composition is characterized in that the concentration of the at least one local anesthetic [LA] is from 1 mg/g to 6 mg/g of total weight of said composition.

In an embodiment, the composition is characterized in that the concentration of the at least one local anesthetic [LA] is from 1 mg/g to 5 mg/g of total weight of said composition.

In an embodiment, the composition is characterized in that the concentration of the at least one local anesthetic [LA] is from 2 mg/g to 5 mg/g of total weight of said composition.

In an embodiment, the composition is characterized in that the concentration of the at least one local anesthetic [LA] is from 6 mg/g to 10 mg/g of total weight of said composition.

In an embodiment, the composition is characterized in that the concentration of the at least one local anesthetic [LA] is 1 mg/g of total weight of said composition.

In an embodiment, the composition is characterized in that the concentration of the at least one local anesthetic [LA] is 3 mg/g of total weight of said composition.

In an embodiment, the composition is characterized in that the concentration of the at least one local anesthetic [LA] is 4 mg/g of total weight of said composition.

In an embodiment, the composition is characterized in that the concentration of the at least one local anesthetic [LA] is 5 mg/g of total weight of said composition.

In an embodiment, the composition is characterized in that the concentration of the at least one local anesthetic [LA] is 6 mg/g of total weight of said composition.

In an embodiment, the composition is characterized in that the concentration of the at least one local anesthetic [LA] is 10 mg/g of total weight of said composition.

In an embodiment, the composition is characterized in that the weight ratio between the concentration of the at least one polyol [Po] and the concentration of the at least one local anesthetic [LA]; [Po]/[LA] is from 0.0002 to 5000; 0.0002≤[Po]/[LA]≤5000.

In an embodiment, the composition is characterized in that the weight ratio between the concentration of the at least one polyol [Po] and the concentration of the at least one local anesthetic [LA]; [Po]/[LA] is from 0.002 to 500; 0.002≤[Po]/[LA]≤500.

In an embodiment, the composition is characterized in that the weight ratio between the concentration of the at least one polyol [Po] and the concentration of the at least one local anesthetic [LA]; [Po]/[LA] is from 0.02 to 50; 0.02≤[Po]/[LA]≤50.

In an embodiment, the composition is characterized in that the weight ratio between the concentration of the at least one polyol [Po] and the concentration of the at least one local anesthetic [LA]; [Po]/[LA] is from 1 to 20; 1≤[Po]/[LA]≤20.

In an embodiment, the composition is characterized in that the weight ratio between the concentration of the at least one polyol [Po] and the concentration of the at least one local anesthetic [LA]; [Po]/[LA] is from 3 to 15; 3≤[Po]/[LA]≤15.

In an embodiment, the composition is characterized in that the weight ratio between the concentration of the at least one polyol [Po] and the concentration of the at least one local anesthetic [LA]; [Po]/[LA] is from 4 to 8; 4≤[Po]/[LA]≤8.

In an embodiment, the composition is characterized in that the weight ratio between the concentration of the at least one polyol [Po] and the concentration of the at least one local anesthetic [LA]; [Po]/[LA] is from 10 to 13; 10≤[Po]/[LA]≤13.

In an embodiment, the composition is characterized in that weight ratio between the concentration of the at least one hyaluronic acid [HA] and the concentration of the at least one local anesthetic [LA]; [HA]/[LA] is from 0.1 to 50; 0.1≤[HA]/[LA]≤50.

In an embodiment, the composition is characterized in that the weight ratio between the concentration of the at least one hyaluronic acid [HA] and the concentration of the at least one local anesthetic [LA]: [HA]/[LA] is from 0.5 to 40, 0.5≤[HA]/[LA]≤40

In an embodiment, the composition is characterized in that the weight ratio between the concentration of the at least one hyaluronic acid [HA] and the concentration of the at least one local anesthetic [LA]: [HA]/[LA] is from 1 to 30; 1≤[HA]/[ LA]≤30.

In an embodiment, the composition is characterized in that the weight ratio between the concentration of the at least one hyaluronic acid [HA] and the concentration of the at least one local anesthetic [LA]: [HA]/[LA] is from 2 to 20; 2≤[HA]/[ LA]≤20.

In an embodiment, the composition is characterized in that the weight ratio between the concentration of the at least one hyaluronic acid [HA] and the concentration of the at least one local anesthetic [LA]: [HA]/[LA] is from 7/3 to 26/3; 7/3≤[HA]/[LA]≤26/3.

In an embodiment, the composition is characterized in that the weight ratio between the concentration of the at least one hyaluronic acid [HA] and the concentration of the at least one local anesthetic [LA]: [HA]/[LA] is from 2 to 20/3; 2≤[HA]/[ LA]≤20/3.

In an embodiment, the composition is characterized in that the weight ratio between the concentration of the at least one hyaluronic acid [HA] and the concentration of the at least one local anesthetic [LA]: [HA]/[LA] is from 2 to 10/3, 2≤[HA]/[ LA]≤10/3.

In an embodiment, the composition is characterized in that the weight ratio between the concentration of the at least one hyaluronic acid [HA] and the concentration of the at least one local anesthetic [LA]: [HA]/[LA] is 20.

In an embodiment, the composition is characterized in that the weight ratio between the concentration of the at least one hyaluronic acid [HA] and the concentration of the at least one local anesthetic [LA]: [HA]/[LA] is 26/3.

In an embodiment, the composition is characterized in that the weight ratio between the concentration of the at least one hyaluronic acid [HA] and the concentration of the at least one local anesthetic [LA]: [HA]/[LA] is 20/3.

In an embodiment, the composition is characterized in that the weight ratio between the concentration of the at least one hyaluronic acid [HA] and the concentration of the at least one local anesthetic [LA]: [HA]/[LA] is 10/3.

In an embodiment, the composition is characterized in that the weight ratio between the concentration of the at least one hyaluronic acid [HA] and the concentration of the at least one local anesthetic [LA]: [HA]/[LA] is 7/3.

In an embodiment, the composition is characterized in that the weight ratio between the concentration of the at least one hyaluronic acid [HA] and the concentration of the at least one local anesthetic [LA]: [HA]/[LA] is 2.

In an embodiment, the composition is characterized in that said composition is sterilized.

In an embodiment, the composition is characterized in that the sterilization is carried out by heat, wet heat, gamma radiation (γ) or accelerated electron beam (electron-beam).

In an embodiment, the composition is characterized in that said sterilization step is carried out by heat.

In an embodiment, the composition is characterized in that the sterilization step is carried out by steam autoclaving.

In an embodiment, the composition is characterized in that the sterilization by steam autoclaving is carried out at a temperature from 121 to 134° C. for a duration adapted to this temperature.

For example, sterilization by steam autoclaving is carried out at a temperature from 127 to 130° C. for a duration from 1 to 20 min.

In an embodiment, the composition is characterized in that the sterilization step is carried out by irradiation with gamma rays (γ).

In an embodiment, the composition is characterized in that the composition moreover includes at least one additional compound.

In an embodiment, the composition is characterized in that the concentration of the at least one additional compound [AC] is from 0.1 to 100 mg/g of total weight of said composition.

In an embodiment, the composition is characterized in that the concentration of the at least one additional compound [AC] is from 1 to 50 mg/g of total weight of said composition.

In an embodiment, the composition is characterized in that the at least one additional compound is dimethyl sulfone, hereafter DMS.

In an embodiment, the composition is characterized in that the at least one additional compound is a water-soluble salt of sucrose octasulfate, hereafter SOS.

In an embodiment, the composition is characterized in that the at least one additional compound is a vitamin C derivative.

In an embodiment, the composition is characterized in that the at least one additional compound is a magnesium ascorbyl phosphate salt, hereafter MAP.

In an embodiment, the composition is characterized in that the at least one additional compound belongs to the family of the catecholamines.

In an embodiment, the composition is characterized in that the at least one additional compound belonging to the family of the catecholamines is epinephrine.

In an embodiment, the composition is characterized in that the concentration of the at least one additional compound [AC] is from 0.01 to 10% by weight with respect to the total weight of said composition.

In an embodiment, the composition is characterized in that the concentration of the at least one additional compound [AC] is from 0.1 to 5% by weight with respect to the total weight of the composition.

In an embodiment, the composition is characterized in that the at least one additional compound is dimethyl sulfone and the concentration thereof is from 1 to 10 mg/g of total weight of said composition.

In an embodiment, the composition is characterized in that the at least one additional compound is a water-soluble salt of sucrose octasulfate and the concentration thereof is from 1 to 40 mg/g of total weight of said composition.

In an embodiment, the composition is characterized in that the at least one additional compound is a magnesium ascorbyl phosphate salt and the concentration thereof is from 0.3 to 20 mg/g of total weight of said composition.

In an embodiment, the composition is characterized in that at least one local anesthetic is released freely in vivo.

### EXAMPLE 1

Two patients (Patient 1 and Patient 2) were monitored with regard to the evolution of the dimensions of their penis as a function of several administrations or implantations by injections. Patient 2 was, in addition, questioned on the satisfaction procured by the treatment, using a scale from 1 to 10.

The injections were administered in different identified zones of the penis, by means of a cannula inserted either at the balanopreputial groove or at the pubic base of the penis.

In general, an initial injection is administered, each zone of the penis being injected with at least 0.15 mL/cm².

As a function of the evolution of the filling in the different zones of the penis body 1, said zones were reinjected or not. In all the cases, each injection is an injection of at least 0.15 mL/cm².

The following table lists the different sizes measured by the practitioner as well as the different volumes injected in the case of patient 1:

TABLE 1
Patient 1
PATIENT 1

| Time (month) | 0 | 1 | 12 | 20 | 31 | 49 | 51 | 72 |
|---|---|---|---|---|---|---|---|---|
| Dimensions before | 110/110 | 125/130 | 130/125 | 140/135 | 140/135 | ND* | 140/155 | 140/145 |
| injection (length/ | | | | | | | | |
| circumference) (mm) | | | | | | | | |
| Total volume injected | 12⁽¹⁾ | 4⁽¹⁾ | 14⁽²⁾ | 6⁽²⁾ | 10⁽³⁾ | 14⁽²⁾ | 0 | 8⁽⁴⁾ |
| (mL) | | | | | | | | |

*ND: not done
⁽¹⁾Formulation used: JUVEDERM 4 ® (concentration of hyaluronic acid: 24 mg/g)
⁽²⁾Formulation used: SURGIDERM 30 ® (concentration of hyaluronic acid: 24 mg/g)
⁽³⁾Formulation used: GLYTONE 4 ® (concentration of hyaluronic acid: 24 mg/g)
⁽⁴⁾Formulation used: STYLAGE L ® (concentration of hyaluronic acid: 24 mg/g)

Comment: during the treatment, even if the sessions are spaced far apart, the dimensions remain greater than the dimensions before the treatment. For example, in month 72, even if no injection had been administered since month 49 (or 23 months without injection), the dimensions (140/145) remain much greater than the dimensions before the treatment, and moreover only the circumference decreased since the measurement of month 51 (only a 10 mm loss in 21 months).

The table below lists the different sizes measured by the practitioner, the volumes injected, as well as the satisfaction of patient 2:

TABLE 2
Patient 2
PATIENT 2

| Time (month) | 0 | 1 | 12 | 13 | 19 |
|---|---|---|---|---|---|
| Dimensions before | 120/95 | 140/120 | 140/115 | 140/125 | 140/120 |
| injection (length/ | | | | | |
| circumference) (mm) | | | | | |
| Total volume injected | 10 | 1 | 8 | 0 | 0 |
| in mL (Formulation used: | | | | | |
| STYLAGE L ®, | | | | | |
| concentration of | | | | | |
| hyaluronic acid: | | | | | |
| 24 mg/g) | | | | | |
| Satisfaction scale (1-10) | *ND | *ND | 8 | *ND | 8 |

*ND: not done

Comment: during the treatment, even if the sessions are spaced far apart, the dimensions remain greater than the dimensions before the treatment. For example, in month 1, the injection of only 1 mL made it possible to almost maintain the dimensions until month 12 (5 mm loss in circumference, and no loss in length in spite of the fact that it had been increased by 20 mm with respect to the length before treatment).

General comment: it was found that the repetition of the injections made it possible to inject less and less hyaluronic acid and/or inject at longer intervals, while keeping the same volume enlarging effect or a greater volume enlarging effect.

1-8. (canceled)

9. A composition including at least one crosslinked hyaluronic acid, which is used in the treatment of locker room syndrome, wherein it is administered at a dose of at least 0.15 mL/cm², and in that said composition is administered repeatedly, and a first administration is followed by n subsequent administration(s) spaced by a time interval from 6 to 15 months, where n≥3.

10. The composition according to claim 9, wherein the dose is at least 0.2 mL/cm².

11. The composition according to claim 9, wherein it is administered in at least one zone of the penis body having an area from 2 to 10 cm².

12. The composition according to claim 9, wherein it is administered in at least one zone of the penis body having an area from 2 to 8 cm².

13. The composition according to claim 9, wherein it is administered in a number of zones of the penis body from 1 to 10.

14. The composition according to claim 9, wherein the concentration of the at least one hyaluronic acid [HA] is from 20 mg/g to 27 mg/g of total weight of said composition.

15. The composition according to claim 9, wherein the hyaluronic acid has an elastic component G′ (25° C., 1 Hz) from 220 to 260 Pa.

16. The composition according to claim 9, wherein the at least one hyaluronic acid has a crosslinking rate X from 0.1 to 0.3.
